(19) United States
(12) Patent Application Publication
(10) Pub. No.: US 2025/0325480 A1
(43) Pub. Date: Oct. 23, 2025

(54) Lipid Nanoparticle Compositions and Methods For Formulating Insoluble Drugs

(71) Applicant: RESPIRERX PHARMACEUTICALS, INC., Glen Rock, NJ (US)

(72) Inventor: Arnold Stan LIPPA, Ridgewood, NJ (US)

(21) Appl. No.: 18/849,500

(22) PCT Filed: Mar. 22, 2023
(86) PCT No.: PCT/US2023/015875

§ 371 Date: Sep. 22, 2024

Related U.S. Application Data
(60) Provisional application No. 63/322,282, filed on Mar. 22, 2022.

(51) Int. Cl. A61K 9/1271; A61K 9/107; A61K 9/51; A61K 31/00; A61K 47/26; A61K 47/44

(52) U.S. Cl. CPC A61K 9/1271; A61K 9/1075; A61K 9/5123; A61K 9/5146; A61K 31/658; A61K 47/26; A61K 47/44

(57) ABSTRACT

The invention provides novel compositions. methods and formulations employing lipid nanoparticle methods and systems for enhancing delivery and bioavailability of poorly soluble drugs, including cannabinoid drugs

### TECHNICAL FIELD

The invention relates to lipid drug formulations, including methods and compositions for selecting and optimizing lipid drug formulations to yield more effective, therapeutic delivery of low solubility drugs, including cannabinoid drugs.

### BACKGROUND

A majority of drug candidates currently under development present bioavailability challenges, leading to difficulties in drug product formulation. These challenges are especially confounding for smaller pharma and biotech companies with limited resources for costly bioavailability enhancement investigation. Several technologies, including particle size reduction, nano-milling, salt formation and amorphous solid dispersions, have been reported to be useful for enhancing oral drug bioavailability. However, these technologies are unpredictable in their implementation and potential efficacy, making them costly and uncertain to investigate. In light of the vast physicochemical diversity of emerging drug candidates, there is an urgent need among drug developers for new technologies to enhance drug delivery and bioavailability.

Low aqueous solubility is a major problem encountered during drug formulation development. Drug candidates that are unable to dissolve in a patient's gastrointestinal tract cannot be systemically absorbed and, as a result, carry a high risk of failure during clinical development. About 70 to 90 percent of drugs in current development fall within low-solubility classes of the Biopharmaceutical Classification System (BCS). It is therefore essential for formulation scientists to discover and employ new solubility enhancement technologies and formulation strategies to improve bioavailability of poorly soluble drugs.

About 70 percent of current drug research pipelines are focused on molecules that are difficult to formulate due to poor or unpredictable bioavailability. These characteristics are often a major cause of product failures. To overcome this problem, lipid-based systems have been investigated to facilitate absorption of active pharmaceutical ingredients (API) and improve bioavailability. However, there are many types of lipid-based systems to investigate, and no single lipid-based formulation can overcome the many, diverse challenges affecting drug solubilization, systemic absorption and processing, and ultimate bioavailability and therapeutic efficacy. Accordingly, drug development companies must often undertake extensive experimentation to identify effective lipid formulations for bioavailability enhancement, which objective remains highly uncertain in its path and prospects for success.

In view of the foregoing, there remains a long unmet need in the art of clinical drug development for more effective compositions and methods to produce and select effective lipid-based drug formulations to enhance clinical delivery, processing and/or bioavailability of insoluble drugs.

### SUMMARY OF EXEMPLARY EMBODIMENTS OF THE INVENTION

The instant invention meets the foregoing needs and satisfies additional objects and advantages, by providing novel compositions and methods for rapid, rational design of lipid-based drug formulations. In exemplary aspects, the disclosure herein describes compositions and methods for manufacturing a variety of lipid nanoparticle (LNP) formulations useful to optimize clinical delivery, absorption, processing and/or bioavailability and therapeutic efficacy for a wide range of low solubility drug candidates, including cannabinoid drug candidates.

### BRIEF DESCRIPTION OF THE DRAWINGS

FIG. 1 is a graphic model illustrating drug solubilization within lipid-based nanoparticulate formulations of the invention (API designated by +symbol).

FIG. 2 is a bar graph illustrating stability results for differentially designed and structured, dronabinol-loaded liposomes. Size and PDI for the liposomes were measured over 7 days of storage. Data are presented according to flow rate ratios (FRRs) and API ratios used to design and construct each of the samples.

FIG. 3 is a bar graph illustrating stability of dronabinol-loaded solid lipid nanoparticles constructed according to the invention. Size and PDI for the solid lipid nanoparticles were measured over 7 days of storage, arranged in the figure according to lipid to API ratios, solid lipid used, and flow rate ratios (FRR) selected to formulate each of the samples.

FIG. 4 is a bar graph illustrating stability of dronabinol-loaded nano emulsions constructed according to the invention. Size and PDI for the nano emulsions measured during 7 days of storage, arranged according to the dronabinol concentrations in mg/ml and the two different total flow rates (TFR) in mL/min that were used to formulate each of the samples.

FIG. 5 illustrates free dronabinol and dronabinol-loaded nanoparticles release profiles. Percent (%) release of dronabinol in the simulated gastric fluid media after 6 hours of incubation. E4 and S4 are the average of 3 samples. * L7 and S3 are averages of 2 samples and for this reason, do not show standard deviation bars.

FIG. 6 illustrates dronabinol loaded nanoparticle release kinetics. Percent (%) release of dronabinol from the selected nanoparticle formulations in the simulated gastric fluid media after 6 hours of incubation. E4 and S4 are the average of 3 samples. * L7 and S3 are averages of 2 samples and for this reason, do not show standard deviation bars.

FIG. 7 graphically illustrates aqueous dispersion stability for dronabinol constructs of the invention. Droplet size and PDI were measured during 7 days of storage at room temperature away from light.

FIG. 8 graphically depicts aqueous dispersion stability post dialysis for novel dronabinol delivery systems of the invention. Droplet size and PDI measured during 7 days of storage at room temperature away from light.

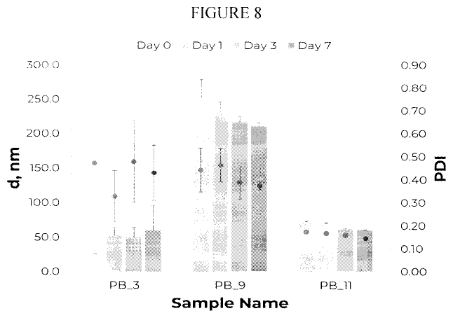

FIG. 9 depicts dronabinol release profiles, expressed as percent (%) release of dronabinol in simulated gastric fluid media after 5 hours of incubation.

### DETAILED DESCRIPTION OF EXEMPLARY EMBODIMENTS OF THE INVENTION

There are many physiological processes, pathways, mechanisms and biochemical and molecular targets within the gastrointestinal tract of mammals where lipid systems integrated into oral drug formulations can interact to facilitate drug delivery, processing and/or bioavailability. When a well-designed lipid formulation enters the GI tract, physiological processes therein can facilitate formation of micro- or nano-emulsions (i.e., emulsions having mean particle sizes in micrometric, or nanometric ranges, respectively). This processing can increase the effective surface area of a lipid emulsion, to enhance drug solubilization, delivery, absorption, processing and/or bioavailability. Once a drug is released from a lipid carrier into the intestinal lumen, maximum solubility of the drug is achieved. enhancing diffusion of the drug through the intestinal membrane. Because drug candidates for lipid-based delivery are provided in a dissolved state in the lipid formulation, no separate dissolution step is required for the drug to become available for absorption.

Many API compounds with low solubility can behave differently if taken with food, which can create variations in PK profile that can lead to adverse side effects. The novel lipid formulations and methods described herein can help to reduce or overcome this food effect.

Lipid formulations and methods of the invention can also aid in circumventing first pass metabolism, which is the action of the intestinal tract to degrade or metabolize APIs by the liver (one of the natural ways the body eliminates compounds). Lipid vehicles produced according to the invention can divert drug delivery via the lymphatic transport system, bypassing liver metabolism. A major factor affecting lymphatic transport is lipophilicity of the API molecule. Drug partitioning into lipid vehicles employing the compositions and methods disclosed herein can enhance lymphatic delivery and/or dosing of lipophilic compounds, bypassing the liver and improving bioavailability.

### Compositions and Methods for Manufacturing Lipid-Based Formulations

The present application describes novel compositions and methods for manufacturing different types of lipid nano-particle (LNP) formulations. The subject compositions and methods are useful to rapidly customize and optimize lipid-based nanoparticulate formulations for specific, low-solubility molecules.

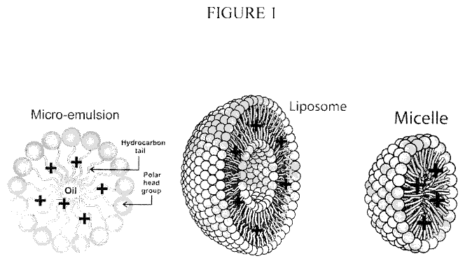

FIG. 1 below depicts drug solubilization within lipid-based nanoparticulate formulations of the invention (the API is designated with a +symbol). In general, nanoparticulate lipid emulsions are clear, thermodynamically stable isotropic liquid mixtures of oil, water and surfactant, often including a cosurfactant. The aqueous phase may contain salt(s) and/or other ingredients, and the “oil” may actually be a complex mixture of hydrocarbons. In contrast to ordinary emulsions, microemulsions form upon simple mixing of the components and do not require high shear conditions generally used in the formation of ordinary emulsions.

A liposome is a spherical vesicle having at least one lipid bilayer. Liposomes are most often composed of phospholipids, especially phosphatidylcholine, but may also include other lipids, such as egg phosphatidylethanolamine, so long as they are compatible with lipid bilayer structure.

A micelle is an aggregate or supramolecular assembly of surfactant phospholipid molecules dispersed in a liquid, forming a colloidal suspension (also known as associated colloidal system). A typical micelle in water forms an aggregate with the hydrophilic “head” regions in contact with surrounding solvent, sequestering hydrophobic single-tail regions in the micelle center.

Solid lipid nanoparticles (sLNP), or lipid nanoparticles (LNPs), are nanoparticles composed of lipids, useful within the invention for pharmaceutical drug delivery. LNPs as a drug delivery vehicle were first approved in 2018 for the siRNA drug Onpattro®. LNPs became more widely known in late 2020, as some COVID-19 vaccines that use RNA vaccine technology coat the fragile mRNA strands with PEGylated lipid nanoparticles as their delivery vehicle (including both the Moderna and the Pfizer-BioNTech COVID-19 vaccines).

Development of solid LNP drug formulations (SLNPs) is an emerging field of lipid nanotechnology, with contemplated applications for clinical medicine and research, as well as in other disciplines. Due to their unique size-dependent properties, lipid nanoparticles offer a possibility to develop or optimize new therapeutics. The ability to incorporate drugs into nanocarriers offers a new paradigm in drug delivery that may hold great promise for enhancing bioavailability of low-solubility drugs, for controlled delivery of drugs, and site-specific drug delivery. SLNP's may also be better tolerated clinically, due to their composition from physiologically compatible lipids.

An exemplary API amenable to delivery and bioavailability enhancement according to the invention is dronabinol, a synthetic Δ9-tetrahydrocannabinol (THC) with direct agonist actions on CB1 and CB2 receptors. Dronabinol was approved in 1986 by the FDA as Marinol® for treating AIDS-related anorexia, and later for treating chemotherapy-induced nausea and vomiting. Additionally, dronabinol has shown clinical efficacy for improving symptoms of sleep-related breathing disorders. Within these clinical uses, dronabinol has presented substantial drug delivery and formulation obstacles, based on the low solubility and poor bioavailability of the drug. A long unmet need therefore exists for new formulations to support these and other indications for dronabinol. A related challenge for dronabinol delivery relates to the unique pharmacokinetic and side effect profiles of this drug. Dronabinol for certain indications, including obstructive sleep apnea (OSA), requires therapeutic blood levels persistent for 4 hours or longer, at levels that do not produce unwanted side effects (see, e.g., U.S. patent application Ser. No. 17/751,508 filed Jan. 9, 2022, and United States Continuation patent application Ser. No. 17/471,102, filed Sep. 8, 2021, each incorporated herein by reference for all purposes).

Dronabinol is commercially formulated today as an oil emulsion in a soft gelatin capsule. Like other hydrophobic molecules, dronabinol presents major drug delivery and bioavailability challenges, including:
a. Dronabinol is water insoluble. This obstacle is currently addressed by formulating the drug as a sesame oil-based emulsion. Absorption of dronabinol in this form (orally administered in a gel-cap) is poor and highly variable, with some patients achieving very high levels, while others achieve very low levels.
b. Dronabinol as presently formulated suffers rapid and extensive metabolic breakdown upon first pass through the liver (approximately 80%), resulting in low blood levels. Additionally, dronabinol has a relatively short half-life (approximately 2-3 hours). Thus, in its current commercial formulation, is poorly suited for therapeutic indications that require therapeutic blood levels to be sustained for 4 hours or longer.
c. In order to achieve effective, sustained therapeutic blood levels to treat OSA and other indications, Applicants have demonstrated that higher or controlled doses of dronabinol are required (see. e.g., U.S. patent application Ser. No. 17/751,508 filed Jan. 9, 2022, and United States Continuation patent application Ser. No. 17/471,102, filed Sep. 8, 2021, each incorporated herein by reference for all purposes). Over an 8-hour test period, 2.5 and 10 mg doses of dronabinol produced therapeutically equivalent OSA alleviating effects for the first 4 hours, but only the 10 mg dose produced therapeutic effects during the second 4 hours. Unfortunately, the 10 mg dose produces unacceptable adverse side effects (Marinol® package insert).

The compositions and methods of the invention overcome the foregoing obstacles to provide effective dosing and delivery of dronabinol and other low-solubility drugs. The tools and processes described herein include novel lipid nanoparticulate dosage forms, along with methods to rapidly design, select and manufacture these dosage forms for therapeutically effective delivery of a wide range of low-solubility drugs.

The following examples illustrate these compositions, methods and articles of manufacture. The skilled artisan will understand that the instant invention is not limited to the particular materials, process steps, and methods of design and use disclosed herein, which are provided for illustrative purposes only. Following the discoveries and teachings of the invention as a whole, the invention can be adapted, optimized and expanded in equivalent form and purpose by the skilled artisan without undue experimentation. Likewise, the terminology employed herein is exemplary to describe illustrative embodiments, and is not intended to limit the scope of the present invention. The following examples are provided for the same, illustrative and non-limiting purpose.

### EXAMPLE I

### Design, Production and Characterization of Lipid Nanoparticle Formulations for Low Solubility Drugs

In the following experiments a series of dronabinol loaded nanoparticles with different excipient systems of various sizes and concentrations were formulated and characterized in order to provide effective LNP-based formulations for dronabinol.

### Experimental Design

Three (3) categories of particles were formulated using dronabinol as the API: 1) liposomes, 2) solid lipid nanoparticles (SLNP) and 3) oil-in-water nano emulsions. Nano emulsions were prepared with water as the aqueous phase, liposomes and SLNPs used phosphate buffered saline (PBS) as the aqueous phase. Other parameters were varied to determine correlation between formulation conditions and physical properties. Lipid to API ratio and flow rate ratio (FRR) were varied for both liposome and solid lipid nanoparticle formulations. For nano emulsions, varied parameters included API concentration and total flow rate (TFR). These variables are summarized in Tables 1-3.

Particle size and polydispersity index (PDI) were measured using a dynamic light scattering (DLS) system post-formulation (day 0), post-dialysis (day 1) and at time points of day 3 (or 4, for nano emulsions) and day 7. Samples were stored at room temperature in clear glass vials in a dark environment. Encapsulation efficiency was calculated based on concentrations of dronabinol in the particle formulations determined via HPLC post-formulation and post-dialysis.

Four exemplary LNP formulations were chosen for dissolution testing to assess dronabinol release in simulated gastric fluid (SGF).

TABLE 1
Liposomes

| ID | Lipid to Drug - L:D | Flow Rate Ratio - FRR | Expected Size | |
|---|---|---|---|---|
| L1 | 10:1 | 1:1 | ~100 | nm |
| L2 | | 2:1 | ~40 | nm |
| L3 | | 3:1 | | |
| L4 | | 4:1 | | |
| L5 | | 5:1 | | |
| L6 | 5:1 | 1:1 | | |
| L7 | | 2:1 | | |
| L8 | | 3:1 | | |
| L9 | | 4:1 | | |
| L10 | | 5:1 | | |

All liposomes were prepared using the same lipid composition of POPC:Chol:DSPE-PEG₂₀₀₀ (72:25:3 mol %) and at a total flow rate of 12 mL/min. Dronabinol concentration varied across the formulations with a maximum predicted value of 1 mg/mL Two sets of formulations were prepared at Lipid to Drug ratios of 10:1 and 5:1, respectively. The aqueous to organic phase flow rate ratio (FRR) varied from 1:1 to 5:1.

TABLE 2
Solid Lipid Nanoparticles

| ID | Lipid to Drug - L:D | Flow Rate Ratio - FRR | Expected Size | |
|---|---|---|---|---|
| S1 | 10:1 | 1:1 | ~200 | nm |
| S2 | 10:1 | 3:1 | ~100 | nm |
| S3 | 10:1 | 5:1 | | |
| S4 | 5:1 | 2:1 | ~sub 80 | nm |

Formulations S1, S2, S3 were made with POPC:Chol:DSPE-PEG₂₀₀₀ (10:89: 1 mol %) lipid composition, at 10:1 lipid to drug ratio, and mixed at a total flow rate of 12 mL/min. The S4 formulation was made using tristearin as the solid lipid with a lipid to drug ratio of 5 to 1. The maximum dronabinol concentration was estimated at 1 mg/mL for all these particles. The flow rate ratio was varied for the different formulations.

TABLE 3
Nano emulsions

| | Total Flow Rate, | Expected | | Target Final THC |
|---|---|---|---|---|
| ID | mL/min | Size | | Concentration, mg/mL |
| E1 | 12 | ~80 | nm | 1 |
| E2 | 12 | ~160 | nm | 2 |
| E3 | 12 | | | 3 |
| E4 | 12 | | | 4 |
| E5 | 12 | | | 5 |
| E6 | 6 | | | 1 |
| E7 | 6 | | | 2 |
| E8 | 6 | | | 3 |
| E9 | 6 | | | 4 |
| E10 | 6 | | | 5 |

Nano emulsions consisted of sesame oil as the carrier oil stabilized with Tween 80 and Span 80 (7:3 ratio) with a total surfactant concentration of 5 mg/mL (HLB 11.8) and surfactant to oil ratio of 1:1. The aqueous to organic flow rate ratio was 1.5:1 for all the formulations. Two sets of formulations were prepared at the total flow rates of 6 mL/min and 12 mL/min, respectively. Target dronabinol concentrations varied from 1 mg/mL to 5 mg/milk

### Materials and Methods

DSPE-PEG₂₀₀₀ (1,2-distearoyl-sn-glycero-3-phosphoethanolamine-N-[methoxy (polyethylene glycol)-2000]) was purchased from Avanti Polar Lipids. Dronabinol was sourced from Toronto Research Chemicals. Transcutol P® was obtained from Gattefossè. HPLC grade solvents were purchased from Fisher Scientific. All other reagents were purchased from Sigma Aldrich and used without purification.

Nanoparticle Preparation Lipids were dissolved in ethanol or Transcutol P as the organic solvent and Calcium-(Ca²⁺) and magnesium- (Mg²⁺) free PBS buffer at pH 7.4, 2.5% Poloxamer 188 or deionized water were used as the aqueous phase. The organic and aqueous phases were rapidly mixed using the Nonensemble Benchtop microfluidic instrument at aqueous to organic Flow Rate Ratios (FRR) between 1:1 and 5:1 and Total Flow Rates (TFR) of 6 mL/min or 12 mL/min, respectively (see Tables 1. 2, 3 for detailed formulation conditions). Formulations were then dialyzed in 10000 MWCO dialysis bags against corresponding aqueous phases to remove ethanol. Liposomal formulations at FRR=1:1 was diluted 2× with corresponding aqueous phase immediately following the mixing process and before dialysis since high amounts of ethanol can destabilize liposomes.
Formulation Stability Samples were stored at room temperature in clear glass vials in a dark environment. The particle size and integrity were measured using Dynamic Light Scattering (DLS) on a Zetasizer, Malvern Instruments, UK, post-formulation (day 0), post-dialysis (day 1) and at time points of day 3 (or 4, for nano emulsions) and day 7. Measurements were taken in triplicate and size and polydispersity index (PDI) are represented as the mean of 3 measurements, and error bars represent standard deviation (SD).
Encapsulation Efficiency Formulation samples (100 uL) were taken post-formulation and post-dialysis, diluted with acetonitrile (900 uL) and sonicated for 15 min to disrupt the nanoparticles and release dronabinol. Dronabinol concentrations were measured by HPLC on the Agilent 1260 Infinity II system with a DAD detector equipped with the Poroshell 120 EC C18 column (2.7 um, 4.6 mm×150 mm). The suspension was quantified in a gradient mode using acetonitrile—water buffered with 0.01% v/v formic acid at a flow rate of 1.1 mL/min.
In Vitro Drug Release Simulated gastric fluid was prepared according to the USP using 3.2 g pepsin, 2.0 g sodium chloride and 7.0 ml of 0.2 M hydrochloric acid per 1 L of the media. Experiments were performed in the sink conditions. Samples from each formulation were placed in dialysis bags and submerged in the simulated gastric fluid (with pepsin) at pH 1.2 and 37° C. in a shaking incubator. Emulsions were dispersed in water as the aqueous phase in the dialysis bags. Liposomes and SLNPs were dispersed in PBS inside the dialysis bags. Aliquots from the media were taken at 5 min, 15 min, 30 min, 1 hr, 2 hrs, 3 hrs, 4 hrs and 6 hrs to assess the amount of dronabinol released from the formulations at each of the time points. Volumes taken were immediately replaced with the fresh SGF to maintain the constant dissolution volume. The amount of dronabinol released into the media for each formulation was measured using ELISA. All the measurements were done in triplicate. THC ELISA kits were purchased from Cayman Chemical. Plates were analyzed on BioTek Epoch 2 microplate spectrophotometer.

### Results and Discussion

Encapsulation Efficiency Encapsulation efficiency (EE) was determined by HPLC analysis. Samples were taken after each formulation and after dialysis to compare the dronabinol concentration between the two. The post-formulation sample yields the total amount of dronabinol (encapsulated and non-encapsulated) and the sample post-dialysis yields just the encapsulated amount of dronabinol, as illustrated by Equation 1, below.

$$EE = - [ [ TTHHC ] ] df \cdot 100 \% , \qquad ( 1 )$$

where EE is the encapsulation efficiency, [THC]_{d} is dronabinol concentration after dialysis and [THC]_{f} is dronabinol concentration post-formulation.

The most stable formulations based on the size and PDI data collected over the 7-day period were then tested to determine their dissolution profiles.

TABLE 4
Encapsulation efficiency of exemplary
dronabinol-loaded liposome formulations

| | HPLC | | |
|---|---|---|---|
| | Post-Formulation | Post-Dialysis | |
| | Dronabinol | Dronabinol | |
| Formulation | conc., mg/mL | conc., mg/mL | EE, % |
| L1 | 0.13 | 0.05 | 34.8 |
| L2 | 0.32 | 0.14 | 44.9 |
| L3 | 0.22 | 0.15 | 68.4 |
| L4 | 0.16 | 0.11 | 71.9 |
| L5 | 0.14 | 0.10 | 71.7 |
| L6 | 0.30 | 0.19 | 64.1 |
| L7 | 0.53 | 0.27 | 51.6 |
| L8 | 0.38 | 0.24 | 63.2 |
| L9 | 0.30 | 0.20 | 66.9 |
| L10 | 0.27 | 0.17 | 62.2 |

Encapsulation efficiency was the highest for the L4 and L5 liposome formulations, which were made using a 10:1 lipid to drug ratio and with the higher FRRs of 4:1 and 5:1, respectively (Table 4). Conversely, the encapsulation efficiency was the lowest for L1 and L2 which were also made with a 10:1 lipid to drug ratio but in the lower range of the FRRs, 1:1 and 2:1 respectively (see, e.g., Table 1 for formulation parameters of specific liposome samples).

Liposomes prepared at 10:1 lipid to drug ratio had overall higher encapsulation efficiencies than the ones prepared at 5:1 lipid to drug ratio. As dronabinol is a hydrophobic compound and is being distributed in the lipid bilayer, higher lipid to drug ratios can yield higher encapsulation efficiencies.

TABLE 5
Encapsulation efficiency of exemplary dronabinol-
loaded solid lipid nanoparticle formulations

| | HPLC | | |
|---|---|---|---|
| | Post-Formulation | Post-Dialysis | |
| | Dronabinol | Dronabinol | |
| Formulation | conc., mg/ml | conc., mg/mL | EE, % |
| S1 | 0.21 | 0.12 | 57.2 |
| S2 | 0.20 | 0.11 | 55.9 |
| S3 | 0.13 | 0.08 | 60.6 |
| S4 | 0.22 | 0.06 | 25.2 |

Encapsulation efficiency was the highest for S3 (Table 5), which was made with cholesterol and at the highest flow rate ratio. All three cholesterol-based formulations (S1-S3) had comparable encapsulation efficiencies of 56-61%; tristearin-based formulation (S4) had considerably lower EE of 25%. Refer to Table 2, for formulation parameters of each solid lipid nanoparticle sample.

TABLE 6
Encapsulation efficiency of the different dronabinol
loaded nano emulsion formulations

| | HPLC | | |
|---|---|---|---|
| | Post-Formulation | Post-Dialysis | |
| | Dronabinol | Dronabinol | |
| Formulation | conc., mg/mL | conc., mg/mL | EE, % |
| E1 | 0.47 | 0.11 | 22.7 |
| E2 | 0.88 | 0.17 | 19.6 |
| E3 | 1.20 | 0.39 | 32.8 |
| E4 | 1.70 | 0.51 | 30.0 |
| E5 | 1.89 | 0.57 | 30.0 |
| E6 | 0.47 | 0.14 | 29.0 |
| E7 | 0.88 | 0.28 | 31.9 |
| E8 | 1.20 | 0.45 | 37.8 |
| E9 | 1.70 | 0.64 | 37.6 |
| E10 | 1.89 | 0.65 | 34.3 |

The encapsulation efficiency was the highest for E8 (Table 6), which was made at the total flow rate of 6 mL/min and with a surfactant to API ratio of 5 to 3. Refer to Table 3, for formulation parameters of each nano emulsion sample.

Encapsulation efficiency of nano emulsions provided within the invention is most affected by total flow rate. Formulations prepared at a lower TFR of 6 mL/min have higher EEs, while formulations prepared at 12 mL/min have lower EEs. This discovery provides for optimization of parameters to achieve nano emulsions with higher EEs.

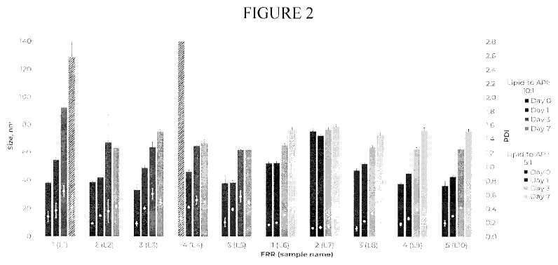

Particle Stability The samples' size and PDI were monitored during 7 days of storage. They were stored at room temperature in clear glass vials in a dark environment. The stability of the particles is determined by the size and PDI change over time. The formulation was considered stable if it maintained a constant size and PDI lower than 0.4. As illustrated in FIG. 2 below, dronabinol-loaded liposome stability was demonstrated to be adjustable according to selectable design and construction parameters.

FIG. 2 presents size and PDI results for exemplary liposome constructs, measured during 7 days of storage, arranged according to the flow rate ratio (FRR) and the two different lipids to API ratios that were chosen to formulate each of the samples. The most stable liposome formulation was L7 which was made with a 5:1 lipid to API ratio and 2:1 flow rate ratio. It showed the smallest size and PDI variation and lowest PDI value within the seven days of storage. This sample was chosen for further dissolution study in simulated gastric fluid conditions. Table 1 provides formulation parameters of each sample, and Table 4 provides respective dronabinol encapsulation efficiency.

Liposome size decreased with increasing flow rate. Increasing the relative amount of the aqueous phase increased the polarity change upon mixing. This, consequently, increased the driving force for the lipid self-assembly into the liposomes and together with the fast mixing at TFR of 12 mL/min limits the local amount of lipids that come together to form a single liposome. This resulted in smaller liposomes at higher FRRs.

Overall, liposomes prepared at 5:1 lipid to drug ratio showed good stability at room temperature and were amenable to provide optimal formulation parameters.

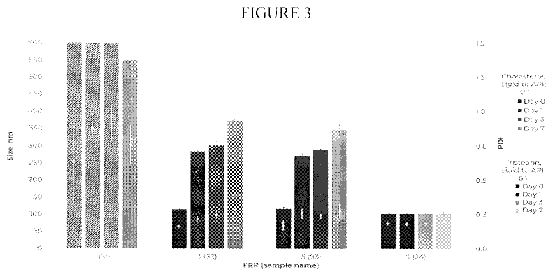

FIG. 3 additionally illustrates stability of uniquely constructed, dronabinol-loaded solid lipid nanoparticles. Size and PDI for the solid lipid nanoparticles measured during 7 days of storage, arranged according to the two different lipids to API ratios, the specific solid lipid used, and the flow rate ratio (FRR) that were chosen to formulate each of the samples.

The most stable dronabinol loaded solid lipid nanoparticle characterized in FIG. 3 was formulation S4, made with tristearin as the solid lipid. This sample shows the least variation in size and PDI up to 7 days of storage. This sample was chosen for further analysis to obtain its dissolution profile in simulated gastric fluid. S2 and S3 were shown to be more stable than S1, with S3 having the higher encapsulation efficiency. S3 was chosen to determine its dissolution profile. Refer to Table 2 for formulation parameters of each sample and Table 5 for their respective dronabinol encapsulation efficiency.

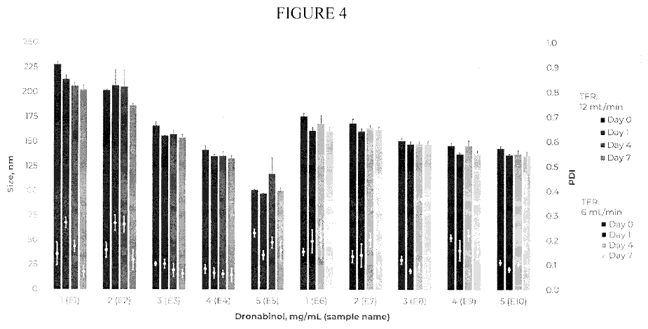

FIG. 4 further illustrates stability findings for dronabinol loaded nano emulsions constructed according to the invention, including to have different total flow rates (TFR) (in mL/min). The most stable dronabinol loaded nano emulsion was E4 which was made at 12 mL/min of total flow rate and using 4 mg/mL of dronabinol. This sample showed the lowest PDI value with minimal variation throughout the 7 days compared to the other samples. This emulsion formulation was chosen for further analysis to obtain its dissolution profile in simulated gastric fluid (refer to Table 3 for formulation parameters of each sample and Table 6 for their respective dronabinol encapsulation efficiency).

Higher dronabinol concentrations allowed for lower droplet size formation which indicates that dronabinol functions as a surfactant and contributes to the stabilization of the system.

### Dronabinol Release Profile

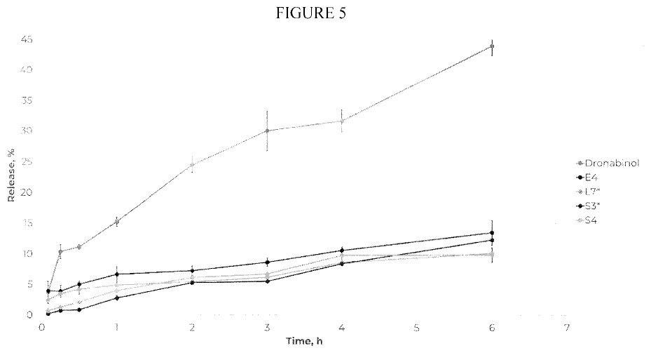

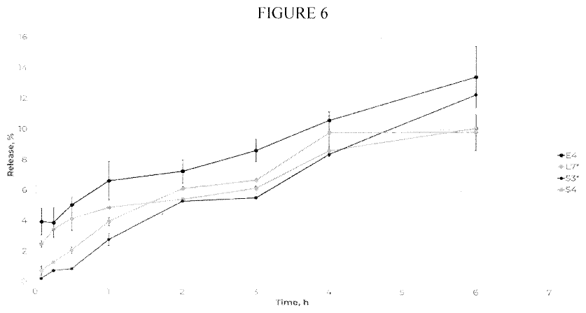

Dissolution studies for nanoparticle constructs of the invention further elucidate the surprising utility of the methods and compositions herein for designing and constructing new dosage forms of insoluble drugs, to provide enhanced delivery, solubility and bioavailability. FIG. 5 depicts differences between free dronabinol (50% ethanolic solution) versus the dronabinol released from the nanoparticles. The results indicated that encapsulated dronabinol was well protected by nanoparticle formulations from the simulated gastric fluid. Over 85% of dronabinol was retained in the nanoparticles during the 6 hours of simulated digestion experiment. This shows that the nanoparticles can potentially reach the small intestine with enough integrity to be taken up by enterocytes and absorbed into the bloodstream where they release the dronabinol at a steady rate. FIG. 6 depicts the difference in the release profiles between the formulations. All four selected formulations retain comparable amounts of dronabinol after the 6 hours of digestion experiment. The emulsion formulation (E4) was less stable in the simulated stomach conditions; however, it did not release more than 14% of the dronabinol.

The foregoing examples demonstrate production and optimization of three series of dronabinol loaded nanoparticles, using optional constructs of liposomes, solid lipid nanoparticles and nano emulsions. Based on the collected stability data, encapsulation efficiency and dissolution profiles, all three systems have potential to be optimized to achieve the target product profile (TPP).

### EXAMPLE II

### Custom Optimization of LNP-Based Dronabinol Formulations

The following studies expand the foregoing discoveries, focusing on optimizing solubilization in various excipient systems and formulations of the invention for the exemplary cannabinoid drug, dronabinol. Different excipients were evaluated individually and in combinations to determine the impact of mixtures on the total dronabinol solubility within the particle. Certain combinations yielded a synergistic solubility effect that dramatically increased solubility in a mixture over that of the individual excipients. All excipients are either FDA approved or GRAS. Phase behavior, particle size, stability and dissolution were also examined.

### Materials and Methods

The following Table illustrates selected excipients used within the instant investigations.

TABLE 7

| Trade name | Chemical name | Type |
|---|---|---|
| Captex 200 | Propylene glycol | Lipid |
| | dicaprylocaprate | |
| Captex 300 | Glyceryl | Lipid |
| | tricaprylate/tricaprate | |
| Tween 80 | Polyoxyethylene (20) sorbitan | Surfactant |
| | monooleate | |
| Span 20 | Sorbital laurate | Surfactant |
| Capmul MCM | Glyceryl caprylate/caprate | Surfactant |
| Propylene glycol | Propylene glycol | Co-solvent |
| Benzyl alcohol | Benzyl alcohol | Co-solvent |
| Dronabinol | (−)-trans-Δ⁹- | Active pharmaceutical |
| | tetrahydrocannabinnol | ingredient (API) |

**Captex 200, Captex 300 and Capmul MCM were obtained from Abitec. Neat Dronabinol and 20% THC in sesame oil were provided by Purisys. Other excipients were purchased from Sigma Aldrich.

Dronabinol quantification by HPLC The sample preparation and HPLC method were as follows. The formulation sample was dispersed into HPLC grade acetonitrile at 0.1 mg/mL concentration and sonicated for 30 min. Dronabinol concentrations were measured by HPLC on the Agilent 1260 Infinity II system with a DAD detector equipped with the Poroshell 120 EC C18 column (2.7 um, 4.6 mm×150 mm). The analyte was quantified in a gradient mode using acetonitrile—water buffered with 0.01% v/v formic acid at a flow rate of 1.1 mL/min.

Aqueous dispersion droplet size and PDI—100 μL of the formulation was dispersed into 10 mL of deionized water and vortexed. The dispersed nanoparticle size and integrity were measured using Dynamic Light Scattering (DLS) on a Zetasizer, Malvern Instruments, UK, post-formulation (day 0), day 1, 3 and 7. Measurements were taken in triplicate and size (intensity weighted mean hydrodynamic size, Z-ave) and polydispersity index (PDI) are represented as the mean of 3 measurements, and error bars represent standard deviation (SD).

Dissolution profile in simulated gastric fluid—Dissolution profiles were created using freshly prepared samples. Dissolutions were performed via the dialysis bag method in simulated gastric fluid (SGF) at pH 1.2 and 37° C. Simulated gastric fluid was prepared according to the USP using 3.2 g pepsin, 2.0 g sodium chloride and 7.0 mL of 0.2 M hydrochloric acid per 1 L of the media. Experiments were performed in the sink conditions. Samples were placed in dialysis bags and submerged in the simulated gastric fluid at pH 1.2 and 37° C. in a shaking incubator. Aliquots from the media were taken at 5 min, 15 min, 30 min, 1 h, 3 h and 5 h to assess the amount of API released from the formulations at each time point. Volumes taken were immediately replaced with fresh media to maintain the constant dissolution volume. Samples were concentrated using Phenomenex Strata® C18-E (55 μm, 70 Å) SPE columns and the API amount was measured by HPLC using the method described above. All the measurements were done in triplicate.

### Results and Discussion

Prepared formulations are summarized in Table 8.

TABLE 8
Dronabinol formulations in the study.

| Sample | | |
|---|---|---|
| name | Excipients | API |
| PB_1 | Captex 300 | Dronabinol |
| PB_2 | Captex 200 | Dronabinol |
| PB_3 | Tween 80 | Dronabinol |
| PB_4 | Span 20 | Dronabinol |
| PB_5 | Capmul MCM | Dronabinol |
| PB_6 | Propylene glycol | Dronabinol |
| PB_7 | Benzyl alcohol | Dronabinol |
| PB_8 | Tween 80:propylene glycol | Dronabinol |
| | 1:1 v/v | |
| PB 9 | Tween 80:Span 20 1:1 v/v | Dronabinol |
| PB_10 | Captex 200:Tween 80:Span | Dronabinol |
| | 20 1:1:1 v/v | |
| PB_11 | Captex 200:Tween 80:Capmul | Dronabinol |
| | MCM 1:1:1 v/v | |
| PB_12 | Tween 80:Span 20 1/1 v/v | 20% THC in sesame oil w/w |

Dronabinol Solubilization—Dronabinol was transferred into pre-weighted 20 mL glass vials with magnetic stirring bars. Corresponding excipients were added to yield 100 mg/mL THC concentration. Samples were stirred at room temperature overnight. Photographs were taken at 30 min after the beginning of the stirring and 24 h timepoints. Dronabinol concentration was determined by HPLC (see Table 2).

TABLE 9
Dronabinol concentration in the experimental samples.

| Sample name | Dronabinol concentration, mg/mL |
|---|---|
| PB_1 | 110.49 ± 0.35 |
| PB_2 | 127.80 ± 0.03 |
| PB_3 | 107.41 ± 0.09 |
| PB_4 | 157.99 ± 0.42 |
| PB_5 | 139.9 ± 0.16 |
| PB_6 | 118.27 ± 0.09 |
| PB_7 | 96.37 ± 0.11 |
| PB_8 | 130.49 ± 0.12 |
| PB_9 | 181.91 ± 0.39 |
| PB_10 | 137.41 ± 0.10 |
| PB_11 | 125.30 ± 0.05 |
| PB_12 | 83.56 ± 0.08 |

Dronabinol was solubilized in all the studied excipients and excipients mixtures in the amount of interest (100 mg/mL) or exceeding it. Solubilization rates varied depending on the excipient. By visual assessment Dronabinol was fully solubilized in PB_2, PB_5, PB_6, PB_7, and PB_11 in 30 minutes or less. PB_1 and PB_8 samples looked uniform after 2 h of mixing on a magnetic stirrer. PB_3, PB_4 and PB_9 demonstrated the slowest rates of solubilization by visual assessment. However, in 24 h of stirring at room temperature all samples appeared uniform, and HPLC results confirmed that Dronabinol fully dissolved in the excipients of choice at a 100 mg/mL concentration.

Aqueous Dispersion Results—Aqueous dispersions of the samples were prepared by adding 100 uL of the stock into 10 ml. of deionized water and vortexing. Dispersed sample behavior was observed and samples that did not show any immediate signs of instability were monitored for droplet size and PDI during 1 week of storage at room temperature away from light. Measurements were taken on days 0, 1, 3 and 7. Sample photographs were taken before vortexing, immediately after vortexing and 24 h after preparation. Observations are summarized in Table 3.

TABLE 10
Sample behavior in aqueous dispersion.

| Sample name | Observations |
|---|---|
| PB_1 | Unstable emulsion; creaming after 24 h |
| PB_2 | Unstable emulsion; creaming after 24 h |
| PB_3 | Clear, no visible phase separation |
| PB_4 | Opaque, no visible phase separation or creaming |
| PB_5 | Opaque, no visible phase separation or creaming |
| PB_6 | Opaque, dronabinol precipitation observed |
| PB_7 | Not miscible; clear with oily film on the surface |
| PB_8 | Opaque, no visible phase separation or creaming |
| PB_9 | Opaque, no visible phase separation or creaming |
| PB_10 | Opaque, no visible phase separation or creaming |
| PB_11 | Translucent, no visible phase separation or creaming |
| PB_12 | Opaque; creaming observed after 24 h |

TABLE 11
Aqueous dispersion sample stability.

| | Day 0 | | | | Day 1 | | | | Day 3 | | | | Day 7 | | | |
|---|---|---|---|---|---|---|---|---|---|---|---|---|---|---|---|---|
| | d, | | | | d, | | | | d, | | | | d, | | | |
| Sample | nm | SD | PDI | SD | nm | SD | PDI | SD | nm | SD | PDI | SD | nm | SD | PDI | SD |
| PB_3 | 12.9 | 0.1 | 0.22 | 0.01 | 16.1 | 0.2 | 0.23 | 0.01 | 18.9 | 0.2 | 0.24 | 0.01 | 23 | 0.2 | 0.30 | 0.03 |
| PB_4 | 5626 | 858 | 0.27 | 0.18 | 4070 | 772 | 0.18 | 0.05 | 5459.7 | 2336 | 0.31 | 0.11 | 1749 | 505.7 | 0.45 | 0.41 |
| PB_5 | 2726 | 915 | 0.65 | 0.33 | 4853 | 1337 | 0.73 | 0.19 | 1859.3 | 517.1 | 0.57 | 0.19 | 1735 | 491.7 | 0.76 | 0.25 |
| PB_8 | 259 | 0.6 | 0.49 | 0.03 | 252 | 2.2 | 0.43 | 0.06 | 250.1 | 8.3 | 0.50 | 0.01 | 243 | 2.5 | 0.45 | 0.06 |
| PB_9 | 282 | 7.4 | 0.42 | 0.01 | 268 | 3.1 | 0.49 | 0.05 | 290.1 | 18.1 | 0.45 | 0.06 | 255 | 8.3 | 0.50 | 0.10 |
| PB_10 | 319 | 20 | 0.49 | 0.02 | 415 | 63 | 0.44 | 0.05 | 424.0 | 34.6 | 0.48 | 0.04 | 560 | 176.3 | 0.56 | 0.06 |
| PB_11 | 60 | 0.5 | 0.18 | 0.01 | 60 | 0.1 | 0.17 | 0.01 | 61.9 | 1.1 | 0.16 | 0.01 | 64 | 1.7 | 0.12 | 0.01 |
| PB_12 | 484 | 72 | 0.50 | 0.02 | 414 | 44 | 0.51 | 0.05 | 603.2 | 105.2 | 0.54 | 0.05 | 689 | 115.7 | 0.65 | 0.12 |

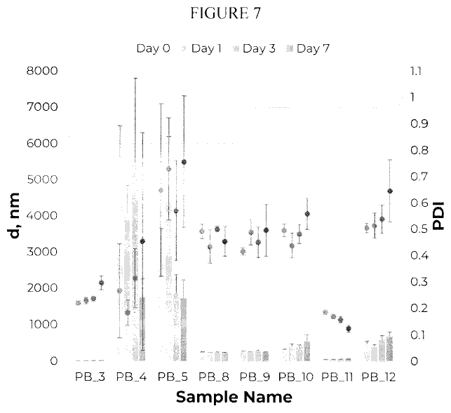

Based on the initial observations, samples PB_3, PB_4, PB_5 and PB_8-PB_12 were monitored for stability during the course of 1 week. Data are shown in the Tables and in FIG. 7 below.

Samples PB_3, PB_9 and PB_11 were chosen for the dialysis test as the most stable based on the size and PDI measurements over 7 days. A 5 mL aliquot of each 7-day old sample was placed in a 10000 MWCO dialysis bag and dialyzed against deionized water overnight to remove any unencapsulated Dronabinol and determine encapsulation efficiency of the corresponding formulation. Dialyzed samples were also stored for the additional 7 days and droplet size and PDI was monitored.

The encapsulation efficiencies (EE %) were determined by HPLC analysis. Samples were taken directly following the formulation step and after dialysis to compare the dronabinol concentration between the two steps and assess EE %. The post-formulation sample yields the total amount of dronabinol (encapsulated and non-encapsulated) and the sample post-dialysis yields only the encapsulated amount of Dronabinol (Equation 1).

$$E = {[ THC ]}_{d} {/ [ THC ]}_{f} \cdot 100 \%$$

where EE is the encapsulation efficiency, [THC]_{d} is Dronabinol concentration after dialysis and [THC]_{f} is dronabinol concentration post-formulation.

TABLE 12
Encapsulation efficiency of dialyzed samples.

| | Day 0 | | | | Day 1 | | | | Day 3 | | | | Day 7 | | | |
|---|---|---|---|---|---|---|---|---|---|---|---|---|---|---|---|---|
| | d, | | | | d, | | | | d, | | | | d, | | | |
| Sample | nm | SD | PDI | SD | nm | SD | PDI | SD | nm | SD | PDI | SD | nm | SD | PDI | SD |
| PB_3 | 26.1 | 0.4 | 0.47 | 0.01 | 53.2 | 11.7 | 0.33 | 0.11 | 49.7 | 14.6 | 0.48 | 0.18 | 59.8 | 36.2 | 0.43 | 0.12 |
| PB_9 | 252.3 | 26.7 | 0.44 | 0.09 | 232.2 | 14.0 | 0.46 | 0.07 | 217.2 | 8.3 | 0.39 | 0.07 | 211.2 | 4.9 | 0.38 | 0.02 |
| PB_11 | 72.0 | 1.4 | 0.17 | 0.01 | 69.9 | 1.5 | 0.17 | 0.00 | 62.4 | 1.2 | 0.16 | 0.01 | 61.1 | 0.3 | 0.15 | 0.00 |

TABLE 13
Aqueous dispersion sample stability post dialysis.

| Sample | THC, mg/mL preD | THC, mg/mL postD | EE, % |
|---|---|---|---|
| PB_3 | 0.5704 | 0.3689 | 64.7 |
| PB_9 | 0.8375 | 0.6590 | 78.7 |
| PB_11 | 0.9640 | 0.9015 | 93.5 |

Changes in the particle size distribution post dialysis in all samples suggest that the smallest particles (<10 nm) are being removed from the sample during the dialysis (Table 6, FIG. 4). PB_3 sample with the lowest average particle size appears affected the most. This agrees with the PB_3 sample having the lowest encapsulation efficiency (Tables).

### Dronabinol Release Profile

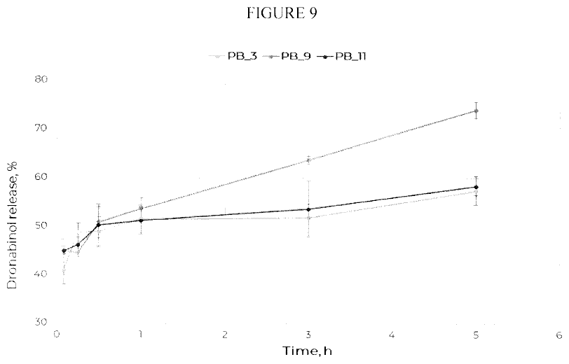

FIG. 9 below depicts the dronabinol release from the aqueous dispersions of samples PB_3, PB_9 and PB_11. Largest particles (PB_9, 250 nm) show faster Dronabinol release than smaller ones (PB_3, 20 nm and PB_11, 60 nm). Table 14 further illustrates Formulated dronabinol stability in simulated gastric fluid.

TABLE 7

| | THC Area % pre-D | THC Area % post-D |
|---|---|---|
| PB_3 | 99.04 | 85.94 |
| PB_9 | 98.66 | 96.55 |
| PB_11 | 96.56 | 95.67 |

Comparison of the THC peak area in the HPLC chromatograms for the pre- and post-dissolution samples taken from inside the dialysis bag indicates that encapsulated Dronabinol can be protected by nanoparticle formulations from the simulated gastric fluid (Table 14).

The foregoing examples illustrate how dronabinol solubility in 12 excipient systems was successfully evaluated toward optimizing clinical delivery and bioavailability of the drug. All excipients and excipient mixtures studied in this scope can be used to obtain Dronabinol solutions of 100 mg/mL Stability of the corresponding aqueous dispersions was assessed, and three formulations with the desirable properties were identified.

Although the foregoing invention has been described in detail by way of example for purposes of clarity of understanding, it will be apparent to the artisan that certain changes and modifications may be practiced within the scope of the appended claims which are presented by way of illustration not limitation. The invention will thus be understood not to be limited, except in accordance to the claims which follow or may later be presented for examination. Various publications and other references have been cited with the foregoing disclosure for economy of description. Each of these references is incorporated herein by reference in its entirety for all purposes.

What is claimed:

1. A pharmaceutical composition comprising a stable lipid nanoparticle construct incorporating a low-solubility active pharmaceutical ingredient (API), wherein the nanoparticle construct is soluble in a physiological aqueous solution.

2. The method of claim 1, wherein the low-solubility API is a cannabinoid compound.

3. The pharmaceutical composition of claim 2, wherein the cannabinoid compound is a dronabinol compound.

4. The pharmaceutical composition of claim 1, wherein the lipid nanoparticle construct has a lipid-API ratio of 10:1 or lower.

5. The pharmaceutical composition of claim 1, wherein the lipid nanoparticle construct has a lipid-API ratio of 10:1 or lower.

6. The pharmaceutical composition of claim 1, wherein the lipid nanoparticle construct has a lipid-API ratio of 5:1 or lower.

7. The pharmaceutical composition of claim 1, wherein the lipid nanoparticle construct has a Flow Rate Ratio (FRR) greater than 2:1.

8. The pharmaceutical composition of claim 1, wherein the lipid nanoparticle construct has a Flow Rate Ratio (FRR) of 4:1 or greater.

9. The pharmaceutical composition of claim 1, wherein the lipid nanoparticle construct has a Flow Rate Ratio (FRR) of 5:1 or greater.

10. The pharmaceutical composition of claim 1, provided in an oral clinical dosage form.

11. A method for manufacturing a pharmaceutical composition comprising a low-solubility active pharmaceutical ingredient (API), comprising incorporating the low-solubility API within a stable lipid nanoparticle construct, wherein the nanoparticle construct is soluble in a physiological aqueous solution.

12. The pharmaceutical composition of claim 11, wherein the low-solubility API is a cannabinoid compound.

13. The pharmaceutical composition of claim 12, wherein the cannabinoid compound is a dronabinol compound.

14. The pharmaceutical composition of claim 11, wherein the lipid nanoparticle construct has a lipid-API ratio of 10:1 or lower.

15. The pharmaceutical composition of claim 11, wherein the lipid nanoparticle construct has a lipid-API ratio of 10:1 or lower.

16. The pharmaceutical composition of claim 11, wherein the lipid nanoparticle construct has a lipid-API ratio of 5:1 or lower.

17. The pharmaceutical composition of claim 11, wherein the lipid nanoparticle construct has a Flow Rate Ratio (FRR) greater than 2:1.

18. The pharmaceutical composition of claim 11, wherein the lipid nanoparticle construct has a Flow Rate Ratio (FRR) of 4:1 or greater.

19. The pharmaceutical composition of claim 11, wherein the lipid nanoparticle construct has a Flow Rate Ratio (FRR) of 5:1 or greater.

20. The pharmaceutical composition of claim 11, further comprising manufacturing the lipid nanoparticle construct in an oral clinical dosage form.
